(19) United States
(12) Patent Application Publication
(10) Pub. No.: US 2006/0276900 A1
(43) Pub. Date: Dec. 7, 2006

(54) Anatomic total disc replacement

(76) Inventor: Clyde T. Carpenter, Olympia, WA (US)

Correspondence Address: Joseph Cagno, Suite B-5, 215 Clay Street NW, Auburn, WA, 98001, US

(21) Appl. No.: 11/142,522

(22) Filed: Jun. 1, 2005

(51) Int. Cl. A61F 2/44

(52) U.S. Cl. 623/17.15; 623/17.16

(57) ABSTRACT

The invention provides an artificial spinal disc prosthesis that can be implanted to replace a damaged natural spinal disc. The implant includes a synthetic polymer ring. The polymers of the ring are oriented along a common pitch angle relative to a common central axis. Orientation of the polymers provides the ring with added strength and durability. Each synthetic polymer ring further comprises an exterior surface and an interior surface thereby forming a hollow area wherein an additional ring or a nucleus may be enclosed. A pair of angulated prosthetic endplates for use with the implant allow for insertion of the device from a multitude of approaches.

### FIELD OF THE INVENTION

This invention relates to an artificial disc which provides for continued mobility and compressibility and which is intended to replace a diseased intervertebral disc.

### BACKGROUND OF THE INVENTION

The vertebrate spine is made of bony structures called vertebral bodies that are separated by soft tissue structures called intervertebral discs. The intervertebral disc is commonly referred to as a spinal disc. The spinal disc primarily serves as a mechanical cushion between the vertebral bones, permitting controlled motions between vertebral segments of the axial skeleton. The disc acts as a synchondral joint and allows physiologic degrees of flexion, extension, lateral bending, and axial rotation. The disc must have mechanical properties to allow these motions and have sufficient elastic strength to resist the external forces and torsional moments caused by the vertebral bones.

The normal disc is a mixed avascular structure comprised of the two vertebral end plates (“end plates”), annulus fibrosis (“annulus”) and nucleus pulposus (“nucleus”). The end plates are composed of thin cartilage overlying a thin layer of hard, cortical bone that attaches to the spongy cancellous bone of the vertebral body. The end plates act to attach the disc to the adjacent vertebrae.

The annulus of the disc is a tough, outer fibrous ring about 10 to 15 millimeters in height and about 15 to 20 millimeters in thickness. The structure of the fibers are like an automobile tire, with 15 to 20 overlapping multiple plies, and inserted into the superior and inferior vertebral bodies at a roughly 30-40 degree angle in both directions. This configuration particularly resists torsion, as about half of the angulated fibers will tighten when the vertebrae rotate in either direction, relative to each other. The laminated plies are less firmly attached to each other. The attached fibers also prevent the disc from extruding laterally with the complex twisting motion of the spine.

Inside the annulus is a gel-like nucleus with high water content. The nucleus acts as a liquid to equalize pressures within the annulus. The material consistency and shape of a normal nucleus pulposis is similar to the inside of a jelly doughnut. The loose fluid-like nature of the nucleus can shrink with compressive forces or swell from osmotic pressure. The ion concentration of the nucleus can create an osmotic swelling pressure of about 0.1 to about 0.3 MPa. As a result, the gel-like nucleus can support an applied load similar to a hydraulic lift. Together, the annulus and nucleus support the spine by flexing with forces produced by the adjacent vertebral bodies during bending, lifting, etc.

The compressive load on the disc changes with posture. When the human body is supine, the compressive load on the third lumbar disc is 300 Newtons (N) which rises to 700 N when an upright stance is assumed. The compressive load increases, yet again, to 1200 N when the body is bent forward by only 20 degrees.

The spinal disc may be displaced or damaged due to trauma or a disease process. A disc herniation occurs when the annulus fibers are weakened or torn and the inner material of the nucleus becomes permanently bulged, distended, or extruded out of its normal, internal annular confines. The mass of a herniated or “slipped” nucleus tissue can compress a spinal nerve, resulting in leg pain, loss of muscle strength and control, rarely even paralysis. Alternatively, with discal degeneration, the nucleus loses its water binding ability and dehydrates with subsequent loss in disc height. Subsequently, the volume of the nucleus decreases, causing the annulus to buckle in areas where the laminated plies are loosely bonded. As these overlapping plies of the annulus buckle and separate, either circumferential or radial annular tears may occur, potentially resulting in persistent and disabling back pain. Adjacent, ancillary facet joints will also be forced into an overriding position, which may cause additional back pain. The most frequent site of occurrence of a herniated disc is in the lower lumbar region. The cervical spinal discs are also commonly affected.

In the United States, low back pain accounts for the most common loss of workdays. Degeneration of an intervertebral disc is one of the most common causes of low back pain and therefore frequently requires treatment. When conservative treatment such as activity modification, medications, physical therapy, or chiropractic manipulation fail, more aggressive measures may be required such as surgical treatment. Spinal fusion has been the mainstay of surgical treatment for recalcitrant low back pain secondary to a degenerated disc. Spinal fusion causes stiffness of the vertebral segment and therefore places increased stresses on adjacent vertebral levels. Replacement of the intervertebral disc with a device that maintains the height of the disc, while still maintaining compressibility and motion is highly desirable and is likely to decrease the back pain associated with a diseased intervertebral disc.

Initial designs of artificial discs were simply a round stainless steel ball intended to replace the painful or herniated intervertebral disc (Fernstrom, J., Acta Chir. Scand. 355:154-9, 1966). This resulted in the steel ball subsiding into the vertebral body and did not maintain disc height nor allow for compressibility. Subsequent designs of intervertebral disc replacements incorporated the ball and socket design but used metal endplates to sit adjacent to the vertebral bodies to prevent subsidence. This ball and socket type design does not allow for a mobile center of rotation in both the axial planes and the sagittal planes. Many of these designs also lack any type of compressible material within the device to absorb compressive forces. See U.S. Pat. No. 4,759,766 (Buttner-Janz); U.S. Pat. No. 5,258,031 (Salib); U.S. Pat. No. 5,246,458 (Graham); U.S. Pat. No. 5,314,477 (Marnay); U.S. Pat. No. 5,425,773 (Boyd); U.S. Pat. No. 5,534,029 (Shima); U.S. Pat. No. 5,676,701 (Yuan); U.S. Pat. No. 5,683,465 (Shinn); U.S. Pat. No. 5,893,889 (Harrington); U.S. Pat. No. 5,895,428 (Berry); U.S. Pat. No. 5,989,291 (Ralph); U.S. Pat. No. 6,113,637 (Gill); U.S. Pat. No. 6,146,421 (Gordon); U.S. Pat. No. 6,179,874 (Cauthen); U.S. Pat. No. 6,368,350 (Erickson); and U.S. Pat. No. 6,540,785 (Gill).

Other designs for artificial disc replacement incorporate some form of compressive springs, see U.S. Pat. No. 4,309,777 (Patil); U.S. Pat. No. 5,320,644 (Baumgartner); U.S. Pat. No. 5,458,642 (Beer); U.S. Pat. No. 5,676,702 (Ratron); U.S. Pat. No. 6,395,032 (Gauchet); and U.S. Pat. No. 6,770,094 (Fehling). This results in motion of metal on metal where the springs are attached to the endplates. This potentially causes release of metal particulate debris into the tissues which can stimulate foreign body reaction (Hallab, N. J., et al., Spine 28:S125-38, 2003). Foreign body reactions can result in resorption of adjacent bone and subsequent subsidence, loosening and pain. Another problem with compressive spring-type prostheses is that they do not resist translational forces well and will eventually fatigue. These devices also lack a mobile instantaneous axis of rotation.

Some current designs use a solid core of elastomeric material, such as polyolefin, to act as a compressible core between two metal endplates, see U.S. Pat. No. 4,349,921 (Kuntz); U.S. Pat. No. 4,863,477 (Monson); U.S. Pat. No. 4,946,378 (Hirayama); U.S. Pat. No. 5,002,576 (Fuhrmann); U.S. Pat. No. 5,071,437 (Steffee); U.S. Pat. No. 5,514,180 (Heggeness); U.S. Pat. No. 5,534,030 (Navarro); U.S. Pat. No. 5,674,296 (Bryan); U.S. Pat. No. 5,824,094 (Serhan); U.S. Pat. No. 6,162,252 (Kuras); U.S. Pat. No. 6,348,071 (Steffee); U.S. Pat. No. 6,419,706 (Graf); and U.S. Pat. No. 6,736,850 (Davis). Devices of this type have the problem of attempting to attach a substance of consistent elasticity to a metal endplate. These types of devices do not resist sheer or translational forces well. One example of this type of device has been implanted in humans and has shown early failures at the elastomeric-metal junction (Fraser, R. D., et al., Spine J. 4:S245-51, 2004).

U.S. Pat. No. 3,867,728, to Stubstad et al., relates to a device, which replaces the entire disc made by laminating vertical, horizontal or axial sheets of elastic polymer. U.S. Pat. No. 4,911,718 to Lee et al., relates to an elastomeric disc spacer comprising three different parts; nucleus, annulus and end-plates, of different materials. Lee teaches a disc made of a specific layered structure of 3-24 separated laminas, unidirectional reinforcing fiber, and specific orientation of these components. The multiple components required in the previous designs by Stubstad et al. and Lee are difficult to fabricate and install, and fail to fully mimic the mechanical dynamics of the normal intervertebral disc, particularly in torsional motion.

### SUMMARY OF THE INVENTION

The foregoing disadvantages of the previously developed prostheses are overcome by providing a novel disc prosthesis that is anatomically configured to fit into the intervertebral disc space after complete debridement of the diseased intervertebral disc. The object of the present invention is to provide a novel spinal disc replacement that is flexible yet strong, can act as a mechanical shock absorber and allow flexibility of motion between the vertebrae. The device is a permanent medical implant for use as a spinal disc. The present invention has elastic moduli that are similar to the normal spinal disc over a range of 0.1 MegaPascals (MPa) to 10 MPa. The elasticity of the present invention allows for shock absorption, flexibility and stability, particularly in torsional motions.

The disc prosthesis comprises at least one polymer ring having a central axis, wherein substantially all the polymers in the ring have the same pitch angle. In preferred embodiments, the disc prosthesis comprises a plurality of polymer rings concentrically arranged with respect to one another, wherein the common pitch angles of at least two rings are different. In preferred embodiments, the common pitch angle in successive concentric polymer rings alternates with one another. While not wishing to be bound by theory, it is believed that the surprising and unexpected properties of the disc prosthesis disclosed herein are due to the oriented polymers in the polymer rings emulating the natural arrangement and anisotropic mechanical properties of type I collagen in the normal annulus fibrosis.

In preferred embodiments, the polymers in the one or more polymer rings comprise ultra-high molecular weight polyethylene (UHMWPE), although other biocompatible polymers may be employed in the instant invention. The macroscopic arrangement of the polymeric material in each ring may take on any form, provided that substantially all the polymers in each ring have the same pitch angle. For example, the UHMWPE in each ring may be macroscopically arranged as either a continuous sheet, a plurality of strands, or as a braided sheet of strands, provided substantially all the polymers in each ring are oriented along the same pitch angle.

In certain embodiments, the one or more polymer rings surround a central hydrogel nucleus that is easily compressible, and in preferred embodiments comprises silicone encapsulated within a soft biocompatible material. In preferred embodiments, the disc prosthesis further comprises two endplates (one superior and one inferior) that are composed of a biocompatible metal such as titanium alloy. The one or more polymer rings are bonded to the endplates by, for example, interdigitations, chemical means, or a compression fitting within the metal endplates.

The position of the endplates relative to one another range from parallel to wedge shaped in order to accommodate to the normal lordotic shape of the spine. In preferred embodiments, the superior and inferior metallic endplates are constructed with a variable height convex surface on their outer wall to accommodate the concavity present in some human vertebral endplates. In yet further embodiments the outer surfaces of the endplates have a porous coating to allow for bone ingrowth into the superior aspect of the superior endplate and into the inferior aspect of the inferior endplate so that the prosthesis attaches biologically to the bone. In preferred embodiments, the porous coating is plasma sprayed with hydroxyapatite to provide for a more rapid biological attachment. In still other embodiments, the endplates also contain a plurality of small teeth (i.e. spikes) which allow for immediate stability after insertion in the intervertebral disc space. In the most preferred embodiments, the outermost polymer ring is covered by a soft elastomeric biocompatible sheet, thereby encapsulating the entire disc prosthesis and providing a single convenient device for replacing a damaged intervertebral disc in a human.

In other aspects of the invention, a disc prosthesis is provided with a plurality of angulated slots on the exterior surface, whereby the angulated slots serve as guides for the implantation of the prosthesis into a patient using different approaches. The approaches range through a full 270 degrees around the spinal axes, and in preferred embodiments comprise anterior, anterolateral, posterolateral, and lateral retroperitoneal approaches. Such flexibility in delivering the prosthesis to the intervertebral space is a great advantage during surgery, where the natural anatomy often limits access to certain disc spaces.

These and other objects of the present invention will become apparent by considering the following drawings and detailed description.

### BRIEF DESCRIPTION OF THE DRAWINGS

The present invention can be better understood by reference to the drawings in which:

FIG. 1 is a perspective view of an illustrative polymer ring according to the present invention illustrating the central axis and central cavity.

FIG. 2 is a series of perspective views of alternative embodiments of illustrative polymer rings according to present invention, where FIG. 2A illustrates an inwardly sloping polymer ring, FIG. 2B illustrates a polymer ring narrower in the center than on the ends, FIG. 2C illustrates a polymer ring wider in the center than on the ends, and FIG. 2D illustrates a slanted polymer ring.

FIG. 3 is an illustrative diagram showing the helical orientation of polymers in a polymer ring and the relationship between their orientation H and the pitch angle λ.

FIG. 4 is a perspective view of an illustrative embodiment of the invention wherein a plurality of polymer rings are arranged concentric to one another to provide a disc prosthesis.

FIG. 5 is an illustrative embodiment of the invention wherein the prosthesis comprises a plurality of concentrically arranged polymer rings sandwiched between two metal endplates and containing a nucleus in their common central cavity. FIG. 5A illustrates the anterior edge view. FIG. 5B illustrates the superior aspect of the prosthesis where the top of the drawing is the posterior edge. FIG. 5C illustrates the inferior aspect of the prosthesis where the top of the drawing is the anterior edge.

FIG. 6 is an exploded view of the illustrative embodiment shown in FIG. 5. FIG. 6A is an exploded view illustrating inferior views. FIG. 6B is an exploded view illustrating superior views.

FIG. 7 is a cross-sectional view of the illustrative embodiment shown in FIG. 5, wherein the view is that designated in FIG. 5B.

FIG. 8 is a perspective view of an illustrative embodiment of the invention wherein the prosthesis is placed using an anterior approach by engaging with a distraction/insertion tool.

FIG. 9 is a perspective view of an illustrative embodiment of the invention wherein the prosthesis is placed using a anterolateral approach by engaging with a distraction/insertion tool.

FIG. 10 is a perspective view of an illustrative embodiment of the invention wherein the prosthesis is placed in a lateral retroperitoneal approach by engaging with a distraction/insertion tool.

### DETAILED DESCRIPTION

While the invention will be described in connection with a preferred embodiment, it will be understood that it is not intended to limit the invention to this embodiment. On the contrary, it is intended to cover all alternatives, modifications and equivalents as may be included within the spirit and scope of the invention as defined by the appended claims.

As used herein, references to certain directions and orientations such as, for example, superior (towards the head), inferior (towards the feet), lateral (towards the side), medial (towards the midline), posterior (towards the back), and anterior (towards the front refer to such directions and orientations in a standing human. As they are applied to embodiments of the invention, it will be further understood that such directions and orientations refer to the position of such embodiments within a human after implantation, wherein the human is standing upright.

Unless specified otherwise, a physical property designated herein for a particular embodiment will be considered to be met provided its value is within 10% of the theoretical value of the physical property. For example, if a theoretical value for the center of mass of an embodiment of the invention is designated to be 10 cm, then it will be understood that embodiments of 9 to 11 cm are within the scope of the invention.

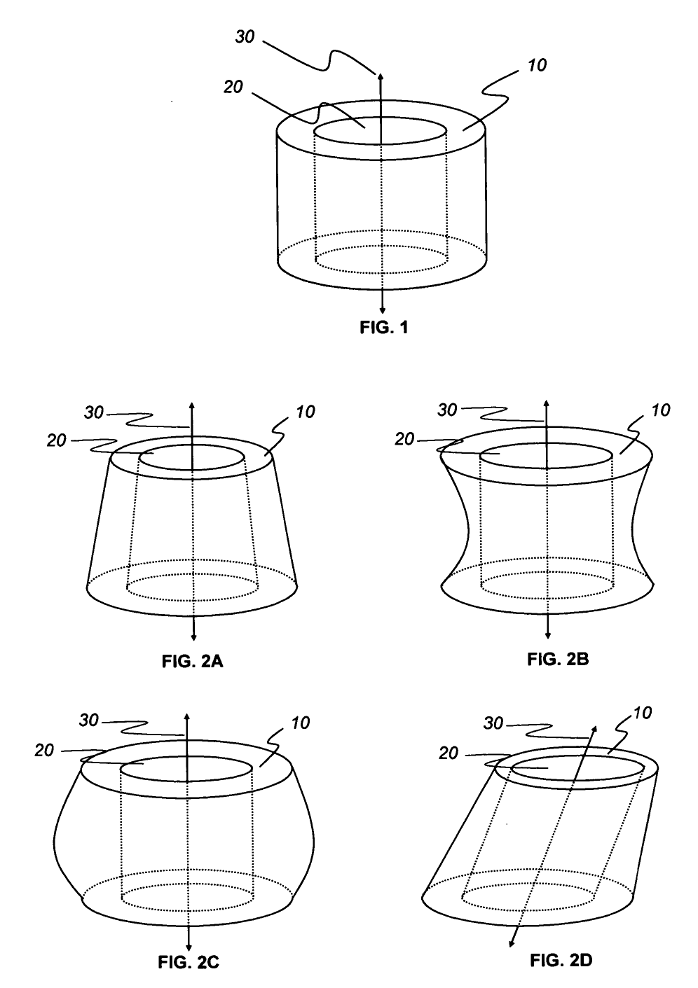

The novel exemplary artificial disc prosthesis comprises in one embodiment, a polymer ring 10 comprising a central cavity 20 and a central axis 30 (FIG. 1). The central axis is defined as an axis which (i) passes only through the empty space of the central cavity, (ii) passes through the center of mass of the polymeric ring, and (iii) adopts a trajectory that results in the greatest rotational inertia (i.e. moment of inertia) of the polymer ring. The definition of center of mass and rotational inertia, as well as methods for calculating their values for any mass body will be familiar to those skilled in the art and available in any common physics textbook (e.g. Fishbane, Gasiorowicz and Thornton, Physics for Scientists and Engineers. Second Edition. Prentice Hall, Upper Saddle River, N.J., 1996).

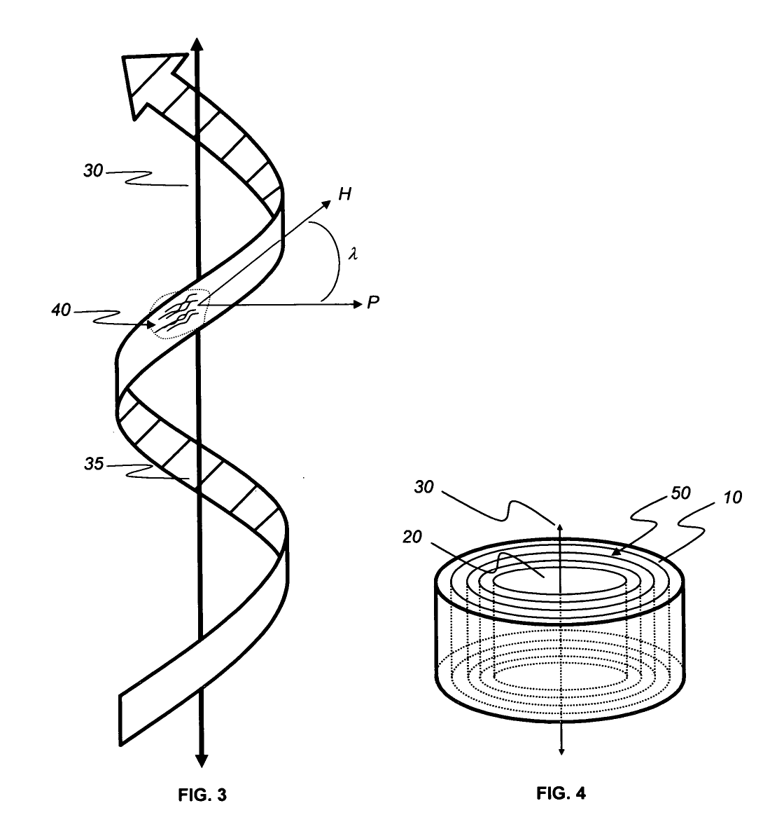

Although preferred embodiments comprise substantially round-cylindrical to ovoid-cylindrical polymer rings, polymer rings of any regular or irregular shape are within the scope of the invention provided they comprise a mass body with a central cavity. For example, other embodiments include polymer rings inwardly sloping toward one end (FIG. 2A), narrower in the center than on the ends (FIG. 2B), wider in the center than on the ends (FIG. 2C), and slanted toward one end (FIG. 2D), wherein each polymer ring 10 comprises a central cavity 20 and central axis 30. As defined herein, inner and outer radii of a polymer ring refer to the distance from a normal to the central axis to the closest and furthest point on the polymer ring, respectively.

A polymer ring comprises a biocompatible polymer having suitable strength and other mechanical properties to resist detrimental spinal movement in the same manner as a natural spinal disc annulus. Thus, in preferred embodiments, the ultimate strength in tension of the polymer ring is generally greater than about 100 kilopascals, and the mechanical elasticity (i.e. Young's modulus and shear modulus) is between 0.1 MegaPascals (MPa) to 100 MPa, and more preferably between 0.2 to 10 MPa. In preferred embodiments the elastic moduli are not constants, but increase with increasing strain. In the most preferred embodiments, the elastic moduli exhibit anisotropy that is dependent on a particular common orientation of polymer molecules within the polymeric ring (see below).

There is sufficient flexibility in the polymer ring to allow at least 2 degrees of rotation between the top and bottom faces of the ring with torsions greater than 0.01 N-m without failing. In preferred embodiments, the polymer ring can withstand compressive loads of at least 100 MPa without failing. This is much more compliant than previously used metals or high molecular weight polyethylene plastics with a compressive modulus typically greater than 100 MPa. The elasticity of the present invention allows for shock absorption and flexibility.

In general, any biocompatible polymer that can be used for biomedical purposes can be used as long as the polymer exhibits a compressive strength of at least 1 MPa, preferably 10 MPa when subjected to the loads of the human spine. The polymer should preferably have an ultimate stretch of 15% or greater, and an ultimate tensile or compressive strength of 100 kilopascals or greater. Hydrophilic polymers are preferred for biocompatibility and controlled swelling characteristics. Methods for identifying polymers and other materials of suitable biocompatibility for use in the invention disclosed herein are well known in the art (e.g. Taksali S, Grauer J N, and Vaccaro A R., Material considerations for intervertebral disc replacement implants. Spine J November-December 2004;4(6 Suppl):231S-238S; Wang Y X, Robertson J L, Spillman W B Jr, and Claus R O. Effects of the chemical structure and the surface properties of polymeric biomaterials on their biocompatibility. Pharm Res. August 2004;21(8):1362-73; and Rizzi G, Scrivani A, Fini M, and Giardino R., Biomedical coatings to improve the tissue-biomaterial interface. Int. J. Artif. Organs. August 2004;27(8):649-57). Biocompatibility may also be defined by cytotoxicity and sensitivity testing specified by ISO (ISO 10993-5 1999: Biological evaluation of medical devices—Part 5: Tests for in vitro cytotoxicity; and ISO 10993-10 2002: Biological Evaluation of medical devices-Part 10: Tests for irritation and delayed-type hypersensitivity).

The polymer ring preferably comprises a polymer material such as ultra high molecular weight polyethylene (UHMWPE), polyethylene, polyamide, polyproplylene, polycarbonate, polysulfone, and other polymers as disclosed in U.S. Pat. No. 4,911,718 to Lee et al., incorporated herein by reference. UHMWPE has a mechanical compressive modulus of elasticity of about 450 MPa, a tensile modulus of elasticity of about 750 MPa, a compressive strength of about 8 MPa, and a tensile ultimate strength of 30 to 40 MPa.

Other polymers useful in the practice of the invention include silicone rubber, polyvinyl alcohol hydrogels, polyvinyl pyrrolidone, poly HEMA, HYPAN™ and Salubria™ biomaterial. Methods for preparation of these polymers and copolymers are well known to the art. In other embodiments the polymer is made of an elastomeric cryogel material disclosed in U.S. Pat. Nos. 5,981,826 and 6,231,605, hereby incorporated by reference, that has a mechanical compressive modulus of elasticity of about 1.0 MPa, ultimate stretch of greater than 15%, and ultimate strength of about 5 MPa. In some embodiments, cryogels may be prepared, from commercially available PVA powders, by any of the methods known to the art. Preferably, they are prepared by the method disclosed in U.S. Pat. Nos. 5,981,826 and 6,231,605, the teachings of which are incorporated herein by reference. Typically, 25 to 50% (by weight) PVA powder is mixed with a solvent, such as water. The mixture is then heated at a temperature of about 100 degrees Celsius (C) until a viscous solution is formed. The solution is then poured or injected into a metal or plastic mold. The mold is allowed to cool to below −10 degree C., preferably to about −20 degree C. The mold is frozen and thawed several times until a solid polymeric ring is formed with the desired mechanical properties. The polymeric ring can them be partially or completely dehydrated for implantation. The resulting disc prosthesis has a mechanical elasticity of 2 MPa and has a mechanical ultimate strength in tension and compression of at least 1 MPa, preferably about 10 MPa. The prosthesis made by this method allows for 10 degrees of rotation between the top and bottom faces with torsions greater than 1 N-m without failing. The device thus made does not fracture when subjected to the same load constraints as the natural intervertebral disc. In some embodiments, the device may be made of a single solid elastomeric material that is biocompatible by cytotoxicity and sensitivity testing specified by ISO (ISO 10993-5 1999: Biological evaluation of medical devices—Part 5: Tests for in vitro (italics) cytotoxicity and ISO 10993-10 2002: Biological Evaluation of medical devices—Part 10: Tests for irritation and delayed-type hypersensitivity.)

In still other embodiments, suitable polymers for use in the polymer ring to achieve the desired range of elastomeric mechanical properties include polyurethane, hydrogels, collagens, hyalurons, proteins and other polymers known to those skilled in the art. Polymers such as silicone and polyurethane are generally known to have mechanical elasticity values of less than 100 MPa. Hydrogels and collagens can also be made with mechanical elasticity values less than 20 MPa and greater than 1.0 MPa. Silicone, polyurethane and some cryogels typically have ultimate tensile strength greater than 100 or 200 kilopascals (KPa). Materials of this type can typically withstand torsions greater than 0.01 N-m without failing.

Although in preferred embodiments, the polymeric ring is a continuous solid comprising substantially a single elastomeric polymer, other embodiments are also within the scope of the invention. Thus, the polymer ring may be constructed of a woven or braided polymer, formed from a fibrous form of the polymer. Weaving or braiding a fibrous form of the polymer material increases the tensile strength and will be known to those of skill in the art of making flexible or expandable polymer containers. In other embodiments, the polymeric ring may be a blend formed from a plurality of polymers. Such polymeric blends often allow the mechanical and elastic properties to be tailored, as the blend will adopt mechanical and elastic values intermediate between the two polymer components. Thus, by varying the relative weight ratio of polymers in the blend, a polymeric ring with a particular sought after set of mechanical and elastic properties can be identified.

In particularly preferred embodiments, substantially all the polymers in the polymeric ring are oriented along a common pitch angle λ. The pitch angle λ is defined herein as the angle made between an axis P within a plane normal to the central axis 30 and an axis H tangent to a spiral arm 35 in the polymeric ring (see FIG. 3). Thus, greater than 70%, 80% and more preferably greater than 90% of the polymers within a particular volume element 40 within the polymeric ring have the same pitch angle λ, where the axis of a particular polymer molecule is defined as the line connecting the first and last atom in the polymer chain. As used herein, “having the same pitch angle λ” means that the pitch angles of the polymers in the ring have a coefficient of variation (CV) less than 40%, less than 30% and more preferably less than 20% (i.e. the CV is the mean pitch angle divided by the standard deviation, where λ is the mean).

Polymeric rings containing oriented polymers exhibit anisotropic elasticity analogous to the native annulus fibrosis, whose collagen polymers are oriented along a common pitch axis. For example, in preferred embodiments the torsional modulus is highest along the axis of polymer orientation H (FIG. 3). Methods for preparing oriented polymers are familiar to those in the art (Ward, I. M., Structure and Properties of Oriented Polymers. Springer, New York, N.Y., USA, 1997; and Ward, I. M., et al., An Introduction to the Mechanical Properties of Solid Polymers. John Wiley & Sons Ltd, West Sussex, England, 2000).

The polymeric ring may be further encapsulated with fibers of polyethylene, a sheet of silicone, polyglycolic acid or poly-paraphenylene terephthalamide, which are arranged in a circumferential direction, preferably as a complete woven mesh ring within the body of the polymeric ring, or as a crisscrossing structure similar to the natural disc annulus. Other methods of encapsulating and reinforcing a polymeric ring of the invention are disclosed in U.S. Pat. No. 4,911,718 to Lee et al., incorporated herein by reference.

In more preferred embodiments, an artificial disc prosthesis according to the present invention comprises a plurality of polymer rings arranged so that they share a common central axis and a common central cavity. In preferred embodiments, a plurality of substantially round-cylindrical to ovoid-cylindrical polymer rings 50 are concentrically arranged so as to have a common central axis 30 and a common central cavity 20 (FIG. 4). By “common central axis” is meant that the axes of each polymer ring do not deviate from one another by more than 20 degrees. In particularly preferred embodiments, the plurality of polymer rings adopt a ‘D’ shape that mimics that shape of the natural annulus fibrosis.

Preferably, from 2 to 20, from 4 to 10, and most preferably 6 to 8 polymer rings are arranged to have a common central axis in order to provide an artificial disc prosthesis according the invention. Each individual ring may be from 0.1 mm to 1 cm thick. Collectively, the exact size of the plurality of polymeric rings can be varied for different individuals. A typical size of an adult disc is 3 cm in the minor axis, 5 cm in the major axis, and 1.5 cm in thickness, but each of these dimensions can vary by 200% without departing from the spirit of the invention.

In preferred embodiments, the disc prosthesis comprises a plurality of concentrically arranged polymer rings wherein the polymer pitch angles of at least two rings are different. In preferred embodiments, the polymer pitch angles of at least two rings differ from one another by at least 10, 20 and most preferably by at least 30 degrees. In preferred embodiments, the polymer pitch angles in successive concentric polymer rings regularly alternate with one another to produce a criss-cross fashion pattern analogous to the rings in the native annulus fibrosis. Thus, in a preferred embodiment, the rings are placed adjacent to each other such that the polymer pitch angle alternates from ring to ring, where a ring with a 30 degree pitch angle is adjacent to a ring with a 150 degree pitch angle which is adjacent to a ring with a 30 degree pitch angle, and so forth. The resulting arrangement thus appears ‘cross-hatched’ when viewed from a lateral perspective. While not wishing to be bound by theory, it is believed that the surprising and unexpected properties of the disc prosthesis disclosed herein are due to the polymeric arrangement in the polymer rings emulating the natural arrangement of collagen polymers in the native annulus fibrosis. In some embodiments, the polymers of the innermost ring adopt a unique orientation, wherein the axes of the polymers are normal to the central axis.

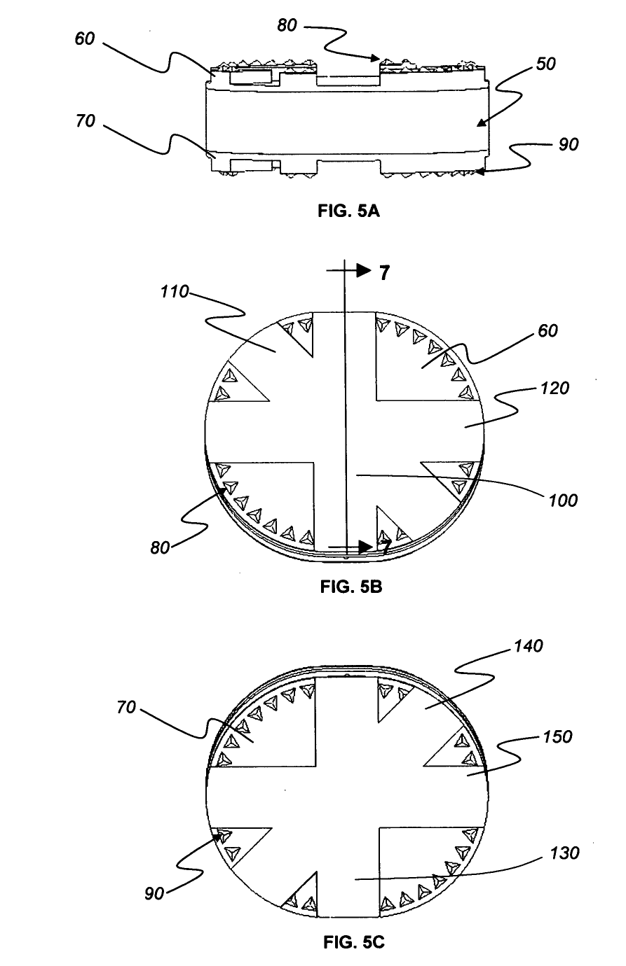

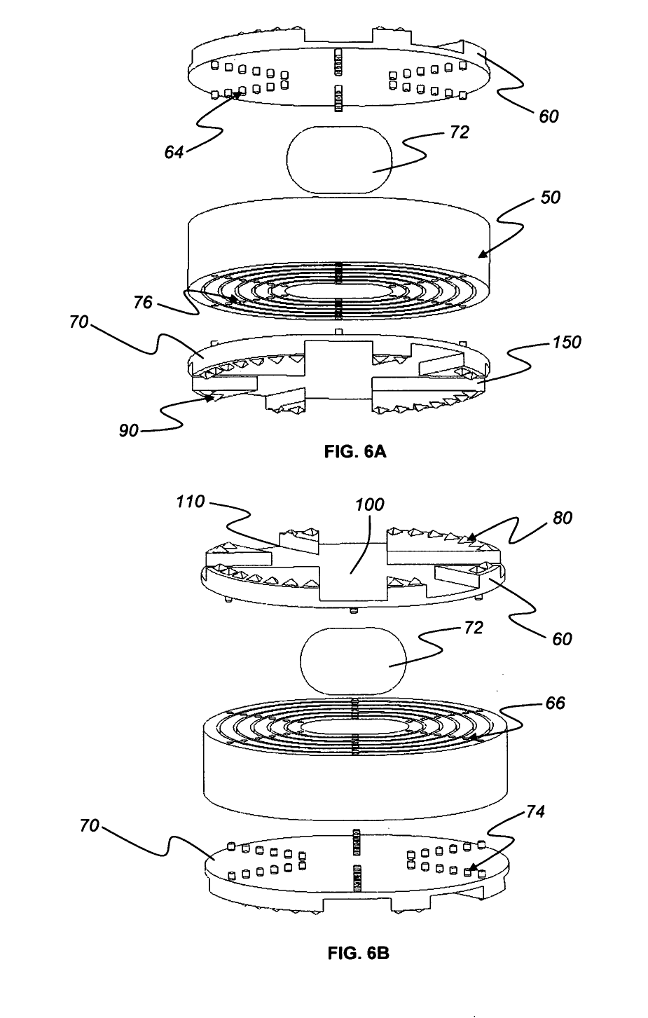

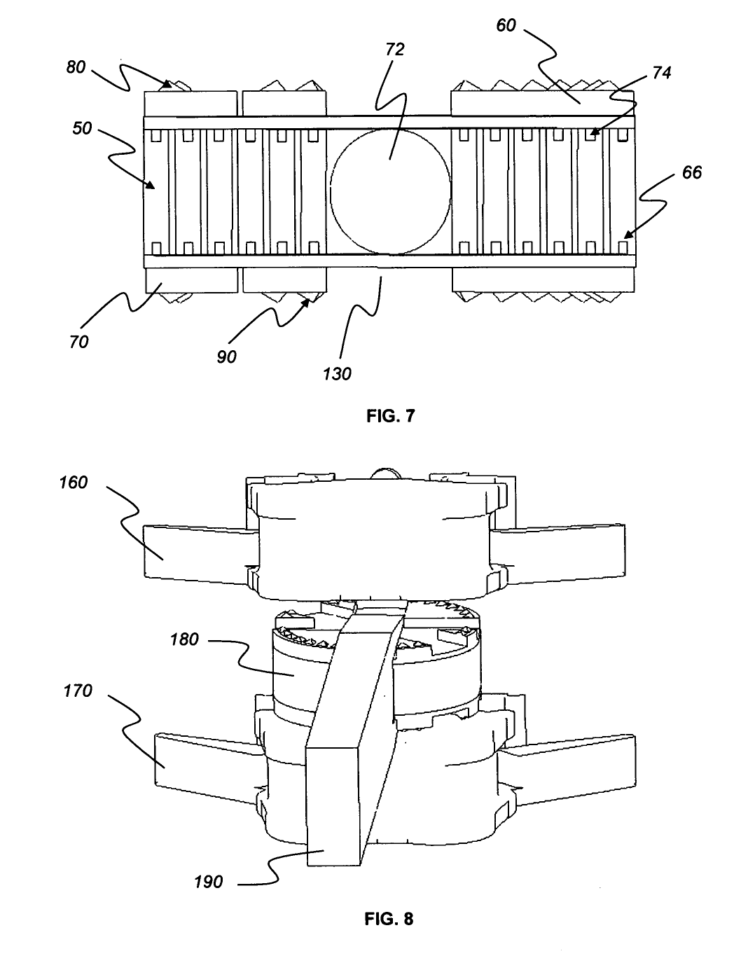

In other preferred embodiments, the disc prosthesis comprises two endplates 60 and 70 that sandwich a plurality of concentric polymer rings 50 having a common central cavity, wherein the central cavity accommodates a hydrogel nucleus 72 (understood by referring to FIGS. 5, 6 and 7). In preferred embodiments, the two endplates are fixedly attached to the plurality of concentric polymer rings via superior interdigitating pegs 64 and holes 66, and inferior interdigitating pegs 74 and holes 76 (FIGS. 6A and 6B). However, it will be appreciated by one skilled in the art that other means of attachment between the endplates and the polymer rings are also possible, and may include for example, the use of adhesive, ultrasonic bonding, melt bonding, epoxy, stitching and any other methods as generally described in U.S. Pat. Nos. 3,867,728 and 4,911,718, incorporated herein by reference. Nucleus 72 is biocompatible (as defined herein), compressible and either free floating or attached to the innermost polymer ring. In preferred embodiments, the nucleus comprises material such as silicon rubbers, hydrogels, polyurethane/silicon composites, and other materials as disclosed in U.S. Pat. Nos. 3,867,728 and 4,911,718, incorporated herein by reference. In use, a compressive force applied to the hydrogel nucleus by opposing superior and inferior forces will result in deformation of the hydrogel nucleus and its outward expansion against the one or more peripheral polymer rings. Thus, the compressive force on the hydrogel nucleus is converted to an outward and tensile force on the one or more peripheral polymer rings. In more preferred embodiments, where the polymer pitch angle alternates from ring to ring, the resulting ‘cross-hatched’ arrangement allows the plurality of polymer rings to withstand unexpectedly large outward forces generated by compression of the central hydrogel nucleus.

The surfaces of the prosthesis in contact with the adjacent vertebral bodies adopt a position relative to one another that ranges from parallel to lordosis in order to accommodate to the relative positions of the superior and inferior vertebrae in the normal spine. In preferred embodiments employing two endplates, the surfaces that adopt a position ranging from parallel to lordosis refer to the endplate surfaces. Further, the surfaces of the endplates are preferably convex in order to accommodate the concavity of some human vertebral endplates. These relative positions and shapes of the prosthesis may be achieved by appropriately shaping the polymer rings, one or both of the endplates, are any combination thereof. In the embodiment illustrated in FIGS. 5, 6, and 7, the surfaces of the prosthesis abutting the adjacent vertebral bodies are substantially parallel to one another.

It is known that a large variation of height, transverse and anterior/posterior size of the disc space, the concavity of the vertebral endplates and the amount of lordosis in the spinal segment exists between humans. Thus, the present invention contemplates the manufacture of prostheses with endplates in a variety of sizes and thicknesses so as to make the necessary selection available to the treating physician to insure a proper fit for a particular patient. In yet other alternative embodiments, the sandwich assembly described above may consist of endplates of a uniform size and instead further comprise a spacer between either the superior or inferior endplate and the plurality of polymer rings. The height of the prosthesis can thus be customized in order to insure accurate anatomic seating of the prosthesis to a particular patient by selecting from a set of spacers having a range of heights.

The endplates in some embodiments are flexible, being comprised of woven or braided metal fibers, wherein the fibers are selected from the group consisting of titanium, aluminum, vanadium, tantalum, cobalt chrome alloy, stainless steel and nitinol. Alternatively, the endplate comprises a polymer or ceramic material in a form that provides a flexible pad having mechanical properties similar to those of a natural spinal disc endplate. In other embodiments the endplates are rigid metal endplates, comprised of a biocompatible metal, such as for example, titanium/aluminum/vanadium alloy, tantalum, cobalt chrome alloy, stainless steel or nitinol. Endplates made from porous titanium is a preferred embodiment. Other materials for the construction of the metal endplates will be familiar to those in the art.

Although in many embodiments, sufficient adhesion can be obtained between the vertebrae and the prosthesis simply by the compressive and frictional forces provided on the prosthesis by the vertebral bodies, in preferred embodiments additional adhesion to the vertebral bodies may be obtained by incorporating surface modifications on the superior and inferior surfaces of the prosthesis that come into contact with the superior and inferior vertebral bodies, respectively. In embodiments employing endplates separate from the one or more polymer rings, it will be understood that surface modifications will be applied to the superior and inferior surfaces of the endplates. In embodiments without endplates, the surface modification will be applied directly to the superior and inferior surfaces of the one or more polymer rings.

The modifications may consist of physical scoring or indentations of the surface, chemical irritants incorporated on the surface, biochemical agents modified on the surface, or small fibers that extend from the faces to stimulate adhesion to a vertebral body or vertebral endplate. These fibers and surface modifications may induce an osteogenic reaction from the person to enhance attachment to the vertebral bodies.

Fixation may be induced by a plurality of methods including open pore or rough surfaces, porous structures with undercuts, incorporation of osteoconductive or inductive agents, incorporation of other polymers such as polyester fabric or fibers, incorporation of other biologically active molecules such as bone morphogenic proteins, tumor necrosis factor or collagen, metal solid or mesh, rough surface with features greater than 5 nanometers. The roughness of the surface may include pores with undercuts of 2 millimeters (mm) in diameter, similar to a sponge. It is anticipated that there are many ways of modifying the surface characteristics of the prosthesis to achieve the same objective of providing cellular in-growth or attachment by collagen or bone. This invention anticipates these factors and others in this class.

One preferred embodiment for mediating adhesion may be understood by referring to FIGS. 5, 6, and 7, which depicts pyramidal surface spikes 80 and 90 approximately 2 mm in height on the superior and inferior endplates, respectively. In other preferred embodiments, the surfaces of endplates 60 and 70 that come in contact with the superior and inferior vertebral bodies comprise porous titanium. The bone-contacting surfaces of the endplates preferably further comprise hydroxyapatite, bone morphogenic proteins, or polycrystalline alumina (Al₂O₃) coatings.

In preferred embodiments, the plurality of polymer rings (in embodiments where no endplates are employed) or endplates (in embodiments where endplates are employed) further comprise a plurality of angulated slots grooved into the exterior surface, wherein each slot on the superior prosthesis surface has a corresponding parallel slot on the inferior prosthesis surface. These angulated slots serve as guides to accurately place the prosthesis between the vertebral bodies using a distraction tool, wherein the slots engage guide rails on the distraction tool to allow the prosthesis to linearly slide into the space formerly occupied by the diseased disc while the distraction tool holds open the disc space. Distraction tools are spreading and insertion forceps well known to those in the art. It will be appreciated that numerous designs of distraction tools are contemplated under the scope of this invention. As defined herein, a distraction tool is a surgical tool that must at least be able to spread adjacent vertebral bodies to expose a disc space and engage the prostheses via one or more exterior slots as described more fully below. Although, prostheses with an anterior to posterior slot are known in the art, the plurality of slots on the endplates described herein permit novel anatomic approaches to place the prosthesis not provided for in the prior art.

In particular, embodiments are contemplated that provide for an anterior to posterior slot, and at least one other slot selected from the set of two slots oriented 45 or 90 degrees to the anterior/posterior slot. In a preferred embodiment, all three slots are provided on endplate 60, thereby providing an anterior to posterior slot 100, a slot 110 oriented 45 degrees to slot 100, and a slot 120 oriented 90 degrees to slot 100 (FIGS. 5A, 5B, 6A, and 6B). Similarly, endplate 70 has three symmetrically placed slots parallel to corresponding slots on endplate 60, thereby providing an anterior to posterior slot 130, a slot 140 oriented 45 degrees to slot 130, and a slot 150 oriented 90 degrees to slot 130 (FIGS. 5A, 5C, 6A, and 6B). Thus, using slot-pair 110 and 140 allows insertion of the prosthesis from an anterolateral approach to the lumbar spine. This approach would be highly advantageous when inserting the device into L4-5 disc space. The L4-5 disc space is bordered anteriorly by the bifurcation of the iliac veins and arteries which make it difficult to obtain direct anterior access to that intervertebral disc space. Additionally, using the slot-pair 120 and 150 allows insertion of the device into the L2-3, L3-4, and possibly the L4-5 intervertebral disc levels from a lateral retroperitoneal flank approach to the spine.

The plurality of polymer rings may optionally further comprise a peripheral elastomeric biocompatible sheet, thereby encapsulating the entire disc prosthesis and providing a single convenient device for replacing a damaged intervertebral disc in a human.

The method manufacture of the above embodiment of the disc prosthesis of the present invention involves at least three separate steps; the first being the preparation of the concentric polymer rings and central nucleus; the second being the fabrication of the endplates; and the third being the assembly of the total prosthesis from these above parts. This assembly can be accomplished in a variety of ways depending primarily on the nature of the constituent materials.

The formation of a polymer ring is dependent upon the particular polymer being utilized. It is envisioned that thermoplastic polymers are preferably used in which case molding under heat and pressure according to the manufacturer's directions may be used to fabricate polymer rings. Methods for manufacturing oriented polymers in the polymer rings are known in the art and will comprise methods as described in Ward, I. M., Structure and Properties of Oriented Polymers. Springer, New York, N.Y., USA, 1997; and Ward, I. M., et al., An Introduction to the Mechanical Properties of Solid Polymers. John Wiley & Sons Ltd, West Sussex, England, 2000, both incorporated herein by reference.

Typical molding, casting and computer-aided machining (CAM) techniques can be used to form endplates. Metallurgical techniques can be used to form metal endplates. Typically, molds are utilized to manufacture prostheses having a geometry consistent with that of a natural disc. Suitable molds can be made from aluminum. Although the disc size can, of course, be varied, a suitable size for the prosthesis is one having a cross section area of 1100 mm², a major diameter of 44 mm and a minor diameter of 30 mm. Both metal endplates and polymer endplates may have porous surfaces or hydroxyapatite surfaces (plasma sprayed) to aid in attachment to adjacent bony vertebral bodies as noted above.

The assembly of the prosthesis typically begins with the formation of a suitably shaped and sized core of concentrically arranged polymer rings of the desired thickness. The nucleus is then placed in the central cavity of the concentrically arranged polymer rings, where the nucleus is manufactured as a hydrogel encased in a membrane or by casting in a metal mold. Where the endplates and polymer rings are attached via interdigitating pegs and holes, respectively, the endplates are then applied with sufficient force to the polymer ring and nucleus assembly to affect attachment. Chemical or other surface modifications of the endplates may be performed before or after assembly of the endplates to the polymer rings. The endplate-polymer ring assembly may then optionally be coated with additional elastomer to encapsulate the final prosthesis.

In use, the prosthesis is delivered using surgical techniques known to those in the art. In one illustrative embodiment where the prosthesis will be placed in the lumbosacral region, the preparation for the retroperitoneal surgery may be the same as in abdominal surgery. A retroperitoneal-anterior or anterior-lateral approach is used to expose the disc spaces. The great vessels and ureters are identified and protected. The anterior longitudinal ligament is incised transversely and opened like a door to expose the injured or degenerated disc. The disc and cartilaginous enplates are removed with a curette, periosteal elevator, chisel, rongueurs, or power drill. Using a distraction tool to apply controlled distraction to the disc space, visualize and remove the remaining disc, thereby performing a complete discectomy. Verify using fluoroscopy that the optimal angle between the bony endplates has been achieved to restore the desired lordosis. With the spacing and degree of lordosis optimized, the prosthesis of the appropriate size is then selected and then loaded onto the guide rails of the distraction tool. Depending on the position of the distraction tool in the patient, the appropriate slot on the prosthesis is engaged onto the distraction tool in order to insure the prosthesis slides into the interverbral space in the correct orientation.

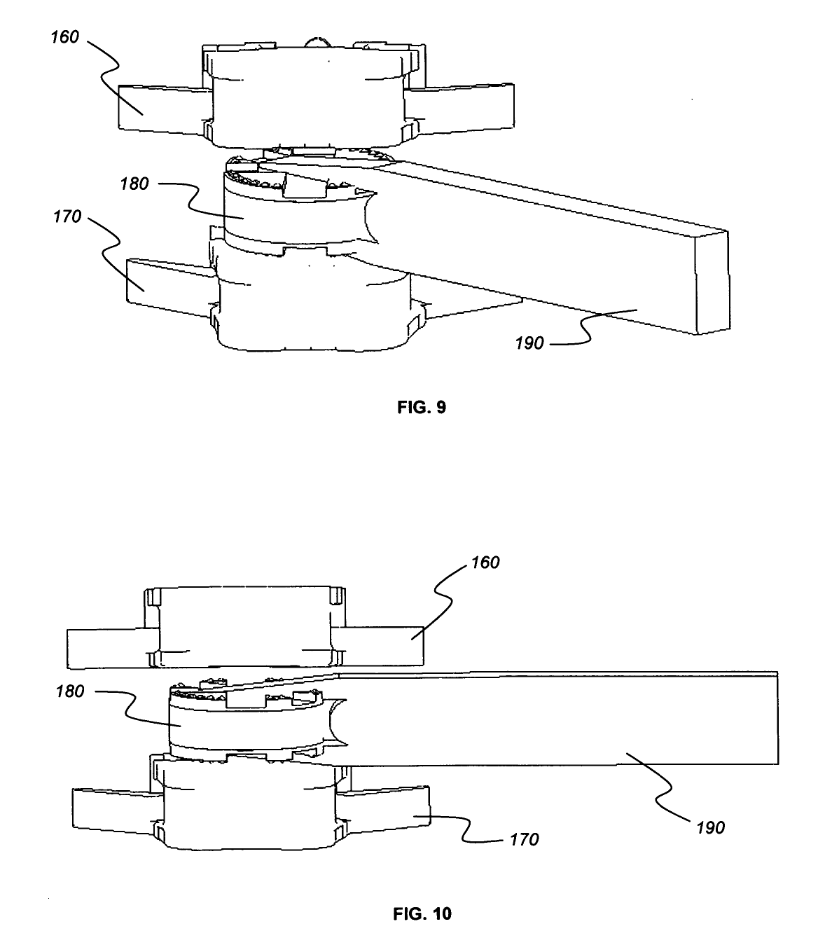

As noted above, the plurality of slots afford multiple approaches for the distraction tool that will allow proper placement of the prosthesis. Thus in certain embodiments, prosthesis 180 of the invention may be placed between vertebrae 160 and 170 using (i) an anterior approach by engaging slot-pair 100 and 130 with a distraction tool 190 (FIG. 8), (ii) an anterolateral approach by engaging slot-pair 110 and 140 with the distraction tool 190 (FIG. 9), and (iii) a lateral retroperitoneal approach by engaging slot-pair 120 and 150 with the distraction tool 190 (FIG. 10). By orienting the distraction tool 180 degrees to the above anterior and anterolateral approaches, it will be further understood that the prosthesis may be placed using (iv) a posterior approach by engaging slot-pair 100 and 130, and (v) a posterolateral approach by engaging slot-pair 110 and 140, respectively (no illustration provided).

In preferred embodiments, the center of the placed prosthesis will correspond to the sagittal midline, and this placement may be verified using lateral and anterior to posterior fluoroscopy. After the proper placement of the prosthesis is confirmed, the spikes on the superior and inferior surfaces of the prosthesis are impacted into the superior and inferior vertebral bodies, respectively. The distraction tool is then released and removed from the prosthesis and disc space. The anterior longitudinal ligament and a portion of the annular ligament, if preserved, are then closed with sutures. The overlying fascia, soft tissue, and skin are closed. The patient is then mobilized.

While several embodiments of the present invention have been described, it is obvious that many changes and modification may be made thereunto without departing from the spirit and scope of the invention.

I claim:

1. A disc prosthesis comprising a polymer ring wherein substantially all the polymers have the same pitch angle.

2. A disc prosthesis according to claim 1 comprising a plurality of concentric polymer rings.

3. A disc prosthesis according to claim 2 wherein the pitch angles of at least two polymer rings are different.

4. A disc prosthesis according to claim 2 wherein the pitch angles of alternating concentric polymer rings are the same.

5. A disc prosthesis according to claim 1, further comprising an endplate.

6. A disc prosthesis according to claim 1, wherein the pitch angle is between 20 and 60 degrees.

7. A disc prosthesis according to claim 1, wherein the disc prosthesis is encapsulated by a biocompatible material selected from the group consisting of polymers, metals, and alloys.

8. A disc prosthesis according to claim 1, further comprising a hydrogel nucleus.

9. A disc prosthesis according to claim 8, wherein a compressive force on the hydrogel nucleus is converted to a tensile force on the polymer ring.

10. A disc prosthesis according to claim 1, wherein the polymer ring is made from materials selected from the group consisting of ultra high molecular weight polyethylene, polyethylene, polyamide, polyproplylene, polyester, polycarbonate, polysulfone, polymethylmethylacrylate, silicone rubber, polyvinyl alcohol hydrogels, polyvinyl pyrrolidone, poly HEMA, HYPAN™, Salubria™, polyurethane, silicone, hydrogels, collagens, hyalurons, and proteins.

11. A disc prosthesis according to claim 1, wherein the polymer ring is made from material in forms selected from the group consisting of a sheet, block, woven polymer fibers, non-woven polymer fibers, mesh, and membrane.

12. A disc prosthesis according to claim 1, wherein the difference between the inner and outer radii of a polymer ring is from 0.01 mm to 100 mm.

13. A disc prosthesis with a plurality of angulated slots on the exterior surface, whereby the angulated slots serve as guides for the implantation of the prosthesis into a patient using different approaches.

14. A disc prosthesis according to claim 13, wherein the angulated slots are on an endplate.

15. A disc prosthesis end plate according to claim 14, wherein the interior surface further comprises a means of attaching to another endplate.

16. A disc prosthesis according to claim 13 wherein the disc comprises an alloy, wherein a metal in the alloy is selected from the group consisting of titanium, aluminum, vanadium, and cobalt.

17. A disc prosthesis according to claim 12, wherein the exterior surface is convex or flat.

18. A disc prosthesis according to claim 13, wherein the exterior surface is coated with a porous ingrowth material.

19. A disc prosthesis according to claim 18, wherein the porous ingrowth material is selected from hydroxyapatite, biochemical agents, small fibers, tumor necrosis factor, and polycrystalline alumina.

20. A disc prosthesis according to claim 13 comprising a plurality of spikes on a surface.

21. A disc prosthesis according to claim 20 wherein the spikes are from 0.5 mm to 10 mm in height.

22. A disc prosthesis according to claim 13 further comprising a means for attachment to a distraction tool.

23. A disc prosthesis according to claim 13 wherein the plurality of angulated slots provide for approaches selected from the group consisting of anterior, anterolateral, posterolateral, and lateral retroperitoneal.

24. A disc prosthesis according to claim 13 further comprising a hydrogel.

25. A disc prosthesis according to claim 13 further comprising a plurality of concentric polymer rings.

26. A disc prosthesis according to claim 13 further comprising a spacer.

27. A disc prosthesis comprising a hydrogel nucleus with means of absorbing compressive force and converting said compressive force to tensile force.
